(19) United States
(12) Patent Application Publication
(10) Pub. No.: US 2001/0007860 A1
(43) Pub. Date: Jul. 12, 2001

(54) METHOD OF DNA VACCINATION

(76) Inventors: CHRISTOPHER W. OLSEN, MADISON, WI (US); WILLIAM F. SWAIN, MADISON, WI (US); DIANE L. LARSEN, SUN PRARIE, WI (US); VERONICA C. NEUMANN, MIDDLETON, WI (US); DAVID P. LUNN, BROOKLYN, WI (US)

Correspondence Address: QUARLES & BRADY LLP, 411 E. WISCONSIN AVENUE, SUITE 2040, MILWAUKEE, WI, 53202-4497, US

(21) Appl. No.: 09/113,836

(22) Filed: Jul. 10, 1998

Related U.S. Application Data
(60) Provisional application No. 60/052,441, filed on Jul. 14, 1997.

(51) Int. Cl. A61K 31/70; C12N 15/63

(52) U.S. Cl. 514/44; 435/320.1; 435/69.1; 435/7.2; 435/69.5; 435/69.52

(57) ABSTRACT

A method of providing a patient with an enhanced immune response is disclosed. In one embodiment, the method comprises the step of vaccinating the patient with a vaccine comprising a combination of DNA encoding interleukin-6 and DNA encoding an antigen capable of enlisting an enhanced immune response in a patient. In one embodiment, the enhanced immune response is a therapeutic response. In another embodiment, the enhanced immune response is a protective immune response.

### CROSS-REFERENCE TO RELATED APPLICATION

This application claims priority to U.S. provisional application 60/052,441 filed Jul. 14, 1997.

### STATEMENT REGARDING FEDERALLY SPONSORED RESEARCH AND DEVELOPMENT

Not applicable.

### BACKGROUND OF THE INVENTION

Influenza A viruses are important pathogens in a variety of mammalian and avian species (Murphy, B. R. and R. G. Webster, 1996). In horses, influenza virus infection is a medically and economically important disease throughout the world and is one of the most common causes of equine respiratory disease in North America (Mumford, J., 1992; Traub-Dargatz, J. L., et al., 1991). Two subtypes of influenza A viruses exist in horses. Viruses of the H3N8 (A/equine-2) subtype are the most commonly isolated subtype throughout the world, whereas H7N7 viruses (A/equine-1) have been identified only rarely in the last 15 years [Murphy, B. R. and R. G. Webster, 1996; Singh, G., 1996; Madic, J., et al., 1996].

Intense vaccination programs for horses are employed in an effort to control infection with influenza virus due to the high morbidity and economic losses associated with outbreaks. However, the inactivated, whole virus vaccines that are commercially-available offer only limited short-term protection (Mumford, J., 1992). In contrast, recovery from natural infection results in complete immunity to reinfection for at least 6 months and partial immunity for over one year (Hannant, D., et al., 1988). Therefore, new vaccination protocols that more closely mimic the responses to natural infection are clearly needed. Mumford and colleagues have shown that ISCOMs prepared from purified equine influenza virus are more highly immunogenic than conventional inactivated virus vaccines and induce protection from challenge in horses (Hannant, D., et al., 1988; Mumford, J. A., et al., 1994), but these vaccines are not available in the U.S. As described below, we have evaluated gene gun-mediated DNA vaccination as an alternative approach. In comparison to administration of preformed protein antigen, DNA vaccination is particularly attractive because the immunogen of interest is actively synthesized de novo in transfected cells. Therefore, the immunogen is available for expression by MHC class I as well as MHC class II molecules (Webster, R. G., et al., 1994; Haynes, J. R., et al., 1996).

DNA vaccination has been shown previously to elicit immune responses to a wide variety of viral, bacterial and protozoal pathogens (Donnelly, J. J., et al., 1994; Sakaguchi, M., et al., 1996; Whalen, R. G., 1996). In particular, immune responses to avian influenza virus infection in chickens and human influenza virus infection in mice and ferrets have been demonstrated following DNA administration via intravenous, intramuscular, intranasal and gene gun-mediated routes of delivery (Webster, R. G., et al., 1994; Ulmer, J. B., et al., 1993; Fynan, E. F., et al., 1993; Ulmer, J. B., et al., 1994). Cutaneous administration of DNA with the gene gun is, however, the most efficient approach, requiring 250-5,000 fold less DNA than parenteral injection techniques (Fynan, E. F., et al., 1995; Pertmer, T. M., et al., 1995). This approach also provides an added safety advantage over intramuscular injection since the administered DNA should be removed from the body through normal epidermal cell turnover (Donnelly, J. J., et al., 1994).

Needed in the art of DNA vaccination is an adjuvant capable of providing an enhanced protective and therapeutic immune response.

### BRIEF SUMMARY OF THE INVENTION

In one embodiment, the present invention is a method of providing a mammalian patient with an enhanced immune response to a specific antigen. The enhanced immune response may be in the form of either protective antibodies or cellular effectors, such as cytotoxic T-lymphocytes (CTL).

The method comprises the steps of vaccinating the patient with a vaccine comprising a combination of DNA encoding interleukin-6 and DNA encoding an antigen capable of eliciting an immune response in the patient. The DNA encoding both the interleukin-6 and the antigen are operably linked to control sequences which direct the expression thereof in the patient.

In one form of the present invention, the interleukin-6 and antigen are encoded on a single nucleic acid construct.

In a preferred form of the invention, the antigen is a viral antigen. Preferably, the virus is one that infects across mucosal surfaces. In a preferred form of the invention, the virus is selected from the group consisting of influenza virus, rotaviruses, herpes viruses and HIV. In another embodiment, the virus may be a blood-borne pathogen, such as hepatitis B virus.

Other forms of the invention encompass antigens specific for bacteria, protozoa, and fungi. Additionally, the present invention is suitable for anti-cancer applications and the antigen is a tumor antigen.

Preferably, the vaccination is via biolistic methods and the preferred vaccination sites are skin and oral or ocular mucosa. An equine vaccination, a preferred site of vaccination is the conjunctiva of the eye.

It is an object of the present invention to provide an enhanced protective antibody immune response.

It is another object of the present invention to provide an enhanced response for cellular effectors, such as CTLs.

It is a feature of the present invention that co-administration of DNA encoding IL-6 with a DNA vaccine enhances the protective immune response of the vaccine.

Other objects, features and advantages of the present invention will become apparent after review of the specification, claims and drawings.

### BRIEF DESCRIPTION OF THE SEVERAL VIEWS OF THE DRAWINGS

FIGS. 1(A) and (B) describe virus-specific serum IgG (A) and IgA (B), as measured by ELISA, in mice vaccinated with control pWRG DNA, Eq/KY HA DNA or Eq/KY HA DNA+ IL-6 DNA. FIG. 1(C) describes virus-neutralizing Ab titers in mice vaccinated with control pWRG DNA, Eq/KY HA DNA or Eq/KY HA DNA+IL-6 DNA.

FIG. 2 shows the ratio of virus-specific IgG1/IgG2a, as measured by ELISA, in the serum of mice vaccinated with control pWRG DNA, Eq/KY HA DNA or Eq/KY HA DNA+IL-6 DNA.

FIG. 3 describes virus-specific IgG titers as measured by ELISA in nasal wash specimens from mice vaccinated with control pWRG DNA, Eq/KY HA DNA or Eq/KY HA DNA+IL-6 DNA.

FIG. 4 shows the mean titers (and standard errors of the means) of virus in the lungs of mice following vaccination with control pWRG DNA, Eq/KY HA DNA, Eq/KY HA DNA+IL-6 DNA or IL-6 DNA alone. FIG. 4(A) shows the results from experiment 1 and FIG. 4(B) shows the results from experiment 2.

### DESCRIPTION OF THE INVENTION

A. Definitions

Unless defined otherwise, all technical and scientific terms used herein have the same meaning as commonly understood by one of ordinary skill in the art to which the invention pertains. The following terms are intended to be defined as indicated below.

An “antigen” refers to any agent, generally a macromolecule, which can elicit an immunological response in an individual. The immunological response may be mediated by B-and/or T-lymphocytic cells. The term may be used to refer to an individual macromolecule or to a homogeneous or heterogeneous population of antigenic macromolecules. As used herein, the “antigen” is generally used to refer to a protein molecule or portion thereof which contains one or more epitopes.

A “B cell epitope” generally refers to the site on an antigen to which a specific antibody molecule binds. The identification of epitopes which are able to elicit an antibody response is readily accomplished using techniques well known in the art. See, e.g., Geysen, et al., 1984) (general method of rapidly synthesizing peptides to determine the location of immunogenic epitopes in a given antigen); U.S. Pat. No. 4,708,871 (procedures for identifying and chemically synthesizing epitopes of antigens); and Geysen, et al., 1986) (techniques for identifying peptides with high affinity for a given antibody).

“T-cell epitopes” are generally those features of a peptide structure capable of inducing a T-cell response. In this regard, it is accepted in the art that T-cell epitopes comprise linear peptide determinants that assume extended conformations within the peptide-binding cleft of MHC molecules, (Unanue, et al., 1987). As used herein, a T-cell epitope is generally a peptide having about 3-5, preferably 5-10 or more amino acid residues.

“Gene delivery” refers to methods or systems for reliably delivering foreign DNA into host cells. Such methods can result in the expression of the foreign DNA in the host cells.

A “nucleotide sequence” or a “nucleic acid molecule” refers to single- or double-stranded DNA and RNA sequences. The term captures molecules that include any of the known base analogues of DNA and RNA.

A “coding sequence” or a sequence which “encodes” a particular polypeptide antigen, is a nucleic acid sequence which is transcribed (in the case of NDA) and translated (in the case of mRNA) into a polypeptide in vitro or in vivo when placed under the control of appropriate regulatory sequences.

The term DNA “regulatory sequences” refers collectively to promoter sequences, polyadenylation signals, transcription termination sequences, upstream regulatory domains, origins of replication, internal ribosome entry sites (“IRES”), enhancers, and the like, which collectively provide for the transcription and translation of a coding sequence in a recipient cell. Not all of these control sequences need always be present so long as the selected gene is capable of being transcribed and translated in an appropriate recipient cell. The control sequences for eukaryotes and prokaryotes can differ significantly, and for the present invention eukaryotic, and preferably, mammalian or mammalian virus control sequences are most suitable.

“Operably linked” refers to an arrangement of elements wherein the components so described are configured so as to perform their usual function. Thus, control sequences operably linked to a coding sequence are capable of affecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

B. General Methods

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention provides a method for eliciting in a mammalian subject, an immune response against a selected antigen using nucleic acid immunization and techniques. The method can thus be used in a variety of mammalian subjects to provide a suitable therapeutic or prophylactic immune response against infection or disease. Suitable antigens include those derived from Human Pappiloma Viruses (HPV), HIV, HSV2/HSV1, influenza virus (types A, B, and C), Polio virus, RSV virus, Rhinoviruses, Rotaviruses, Hepatitis A virus, Norwalk Virus Group, Enteroviruses, Astroviruses, Measles virus, Para Influenza virus, Mumps virus, Varicella-Zoster virus, Cytomegalovirus, Epstein-Barr virus, Adenoviruses, Rubella virus, Human T-cell Lymphoma type I virus (HTLV-I), Hepatitis B virus (HBV), Hepatitis C virus (HCV), Hepatitis D virus, Px virus, Marbug and Ebola; bacteria including M. tuberculosis, Chlamydia, N. Gonorrhea, Shigella, Salmonella, Vibrio Cholera, Treponema pallidua, Pseudomonas, Bordetella pertusis, Brucella, Franciscella tulorensis, Helicobacter pylori, Leptospria interrogaus, Legionella pneomophila, Yersinina pestis, Streptococcus (types A and B), Pneumococcus, Meningococcus, Hemophilus influenza (type B), Toxoplasma gondic, Complylobacteriosis, Moraxella catarrhalis, Legionella pneumophlia, Pseudomonas aeruginosa, Donovanosis and Actinomycosis; fungal pathogens including Candidiasis and Aspergillosis, parasitic pathogens including Taenia, Flukes, Roundworms, Amebiasis, Giardiasis, Cryptosporidium, Schistosoma, Pneumocystis carinii, Trichomoniasis and Trichinosis. The present invention can be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral virus diarrhea (BVDV), Klebsiella pneumoniae, E. coli, Bordetella pertussis, Bordetella parapertussis and brochiseptica.

C. Isolation of Genes and Construction of Vectors

Nucleotide sequences selected for use in the present invention can be derived from known sources, for example, by isolating the same from infected cells or viral particles containing a desired gene or nucleotide sequence using standard techniques. The nucleotide sequences for many, if not most, pathogen antigens have been identified to assist in vaccine and therapy design. It is now possible to construct DNA molecules of significant length once DNA sequence information is available.

Once coding sequences for desired antigens have been prepared or isolated, such sequences can be cloned into any suitable vector or replicon. Numerous cloning vectors are known to those of skill in the art, and the selection of an appropriate cloning vector is a matter of choice. Ligations to other sequences are performed using standard procedures, known in the art.

Selected nucleotide sequences can be placed under the control of regulatory sequence such as a promoter or ribosome binding site (also referred to herein as “control” elements), so that the sequence encoding the desired antigen is transcribed into RNA in the host tissue transformed by a vector containing this expression construct.

The choice of control elements will depend on the host being treated and the type of preparation used. Thus, if the host's endogenous transcription and translation machinery will be used to express the proteins, control elements compatible with the particular host will be utilized. In this regard, several promoters for use in mammalian systems are known in the art and include, but are not limited to, promoters derived from SV40, CMV, HSV, RSV, MMTV, among others.

In addition to control sequences, it may be desirable to add regulatory sequences which allow for regulation of the expression of antigens encoded by the delivered nucleotide sequences. Regulatory sequences are known to those of skill in the art, and examples include those which cause the expression of a coding sequence to be turned on or off in response to a chemical of physical stimulus, including the presence of a regulatory compound. Other types of regulatory elements may also be present in the vector, for example, enhancer sequences.

An expression vector is constructed so that the particular coding sequence is located in the vector with the appropriate control and, optionally, regulatory sequences such that the positioning and orientation of the coding sequence with respect to the control sequences allows the coding sequence to be transcribed under the “control” of the control sequences (i.e., RNA polymerase, which binds to the DNA molecule at the control sequences, transcribes the coding sequence). Modification of the sequences encoding the particular antigen of interest may be desirable to achieve this end. For example, in some cases it may be necessary to modify the sequence so that it is attached to the control sequences with the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

Conventional mammalian expression vectors and elements can be enhanced for use as DNA vaccines. For example, it has been found that the addition of signal peptide sequences directing secretion of expressed proteins can enhance CTL immune response. The use of a Kozak ATG sequence can enhance the translational efficiency of a DNA vaccine. The inclusion of a mono/poly ubiquitination sequence in the expression vector can enhance the MHC Class I presentation signal while alternatively the use of an invariant chain sequence can enhance MHC Class II presentation signal. The use of such elements is within the abilities of those of skill in the art.

D. Administration of Nucleic Acid Preparations

Particle-mediated methods for delivering nucleic acid preparations are known in the art. Thus, once prepared and suitably purified, the above-described nucleic acid molecules can be coated onto carrier particles using a variety of techniques known in the art. Carrier particles are selected from materials which have a suitable density in the range of particle sizes typically used for intracellular delivery from a gene gun device. The optimum carrier particle size will, of course, depend on the diameter of the target cells.

For the purpose of the invention, tungsten, gold, platinum and iridium carrier particles can be used. Tungsten and gold particles are preferred. Tungsten particles are readily available in average sizes of 0.5 to 2.0 um in diameter. Although such particles have optimal density for use in particular acceleration delivery methods, and allow highly efficient coating with DNA, tungsten may potentially be toxic to certain cell types and may degrade DNA over time. Gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 um, or available from Degussa, South Plainfield, N.J. in a range of particle sizes including 0.95 um) and reduced toxicity. Microcrystalline gold provides a diverse particle size distribution, typically in the range of 0.5-5 um.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the-coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particular gene gun instruments.

E. Administration of Coated Particles

Following their formation, carrier particles coated with either nucleic acid preparations, or peptide or protein antigen preparations, are delivered to mucosal tissue using particle-mediated delivery techniques.

Various particle acceleration devices suitable for particle-mediated delivery are known in the art, and are all suited for use in the practice of the invention. Current particle acceleration device designs employ an explosive, electric or gaseous discharge to propel coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is the instrument (PowderJect Vaccines, Inc., Madison, Wis.), which instrument is described in U.S. Pat. No. 5,120,657. Another electric discharge apparatus suitable for use herein is described in U.S. Pat. No. 5,149,655. The disclosure of all of these patents is incorporated herein by reference in their entireties.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 10.0 ug, more preferably 0.25 to 5.0 ug of nucleic acid molecule per dose, depends on the subject to be treated. By dose, it is meant to refer to a single event of delivery, for example, by gene gun. Using current gene guns, it is common for a single immunization procedure, whether a prime immunization or a boost, to include more than one gene gun dose. For example, a prime might consist of two to six gene gun doses to the tongue. Adding more DNA to each dose, beyond 0.25 to 5 ug, generally does not increase immune response. The additional doses are appropriate to, in essence, treat more tissue. A gene gun design which is capable of treating more tissue in a single operation would lower the number of doses in single vaccination. In general, however, the total amount of DNA delivered in the entire immunization will be in the range of about 1-30 ug total for all doses. Often a prime immunization and either one or two boost immunizations will be appropriate to achieve the desired level of immune response. The exact amount necessary will vary depending on the age and general condition of the individual being immunized and the particular nucleotide sequence or peptide antigens selected, as well as other factors. An appropriate effective amount can be readily determined by one of skill in the art upon reading the instant specification.

F. In General

The experiments described below were conducted to investigate whether co-administration of DNA encoding human interleukin-6 (IL-6) would enhance immune responses to a hemagglutinin (HA) or hepatitis DNA vaccine in model mouse systems for both viruses and an equine system for influenza virus. The experiments showed that influenza virus-specific serum IgA and nasal IgG appeared more rapidly and serum virus neutralizing antibody titers were higher prior to challenge in the mice vaccinated with HA+IL-6 DNA compared to those that received HA DNA alone. In horses, co-administration of HA and equine IL-6 DNA enhanced influenza virus-specific serum IgG antibody responses with a broadening of the responses to include the IgG(T) isotype, IgA responses in nasal secretions, and lymphocyte proliferation responses in the lymph nodes draining the site of DNA vaccination.

In a similar fashion, mice vaccinated with a hepatitis B virus DNA vaccine and IL-6 DNA demonstrated enhanced immune responses compared to those receiving hepatitis B virus DNA alone. Specifically, there was an increase in CTL responses with mice immunized by co-delivery of IL-6.

Following homologous influenza virus challenge infection, mice that had received HA DNA alone had reduced levels of virus in their lungs and cleared their infections more rapidly than controls, but they were not protected from infection. In contrast, mice that received both HA and IL-6 DNA were protected, as evidenced by a lack of detectable virus in their lungs after challenge. These results support the use of IL-6 as a cytokine adjuvant to DNA vaccination.

Therefore, in one embodiment, the present invention is a method of enhancing protective immune responses by co-administering DNA encoding interleukin-6, with a DNA vaccine. This protective immune response is most easily measured by virus-specific antibody responses in serum. or in mucosal secretions and by prevention or reduction of virus replication.

The method of the present invention provides that following virus infection, the patients receiving both the DNA vaccine and IL-6 will have reduced detectable virus, preferably tested in lungs for influenza virus. Most preferably, the vaccine recipient will be completely protected as evidenced by a total lack of detectable virus.

Our results below also show that the vaccinated patient will show an increased cytotoxic T-lymphocytes (CTL) levels. Our Examples below demonstrate that levels of CTL are greater than or equal to 45% higher in mice immunized with the vaccine plus IL-6 than in mice immunized with only the vaccine.

Preferably, the method of vaccination is via particle-mediated gene transfer methods, such as the PowderJect gene delivery device. Other methods of DNA vaccination suitable for the present invention are summarized in Fynan, E. F., et al., 1993; Donnelly, J. J., et al.,1994; Fynan, E. F., et al., 1995; Liu, M. A., 1995 (entire volume was devoted to DNA vaccination); Robinson, H. L., et al., 1996; Ulmer, J. B., et al., 1996; and Donnelly, J. J., et al., 1996).

Preferred sites of immunization are the skin surface of the patient and the oral or ocular mucosal surfaces.

Preferably, the DNA vaccine comprises an antigen capable of eliciting a protective immune response in the patient.

In one preferred embodiment, the DNA vaccine would comprise the influenza hemagglutinin gene, the hepatitis surface antigen, or other genes from viruses that infect across mucosal surfaces. Therefore, we envision that viruses such as influenza viruses, HIV, rotaviruses and herpes viruses are especially suitable for the present invention.

In another embodiment of the present invention, one may wish to immunize against blood-borne pathogens, such as hepatitis B virus.

In another embodiment, the antigen is specific for a bacterial, protazoan, or fungal protein and the vaccine is designed to elicit immune response against these pathogens.

In another embodiment of the invention, the antigen is designed to provoke an anti-cancer response. Examples of such an antigen are gp100, Mart-1, Epcam-1 and MuC-1. In this embodiment of the present invention, one would immunize a tumor patient and invoke a therapeutic CTL response.

We envision that mammals, such as horses, pigs, cows, and humans, and avian species, such as birds, can be successfully vaccinated by the present invention.

Preferably, one would use a human interleukin-6 gene when vaccinating humans and an equine interleukin-6 gene when vaccinating horses. However, our studies show that heterologous genes are also useful.

The examples below disclose preferred methods of co-administering the IL-6 DNA and vaccine. Other methods of preparing these reagents will be apparent to one of skill in the art of microbiology.

### EXAMPLES

1. Use of DNA Encoding Human IL-6 as an adjuvant to influenza virus HA DNA Vaccination in Mice (as described in Larsen, et al., 1998, incorporated by reference)

A. In General

We have chosen the hemagglutinin (HA) protein as our immunogen because it is the viral protein that contains the major antigenic sites of the virus and it is the protein to which virus neutralizing (VN) antibodies are directed. The HA protein is responsible for virus binding to cells and fusion of the virus envelope and cell membrane to initiate infection (Murphy, B. R. and R. G. Webster, 1996). We have demonstrated previously that ACCELL gene gun-mediated DNA vaccination using the HA gene of A/Equine/Kentucky/1/81 (H3N8) (Eq/KY) virus induces virus-specific antibodies (Abs), including VN Abs, in mice (Olsen, C. W., et al., 1997). However, only partial protection from challenge infection was achieved unless a very prolonged time period was provided between doses of vaccine (Olsen, C. W., et al., 1997). In contrast, recovery of mice from a previous infection with Eq/KY virus conferred complete immunity to homologous virus challenge (Olsen, C. W., et al., 1997).

In an attempt to mimic the host response to natural infection, we hypothesized that addition of a cytokine adjuvant would enhance the immune responses generated by our HA DNA vaccine and subsequent protection from infection. Previous studies have investigated the use of cytokines as vaccine adjuvants. Interleukins-1, -3, -4, -6, -7 and -12, as well as IFN-gamma, GM-CSF and TNF-alpha have been administered in protein form (Lin, R., et al., 1995; Lofthouse, S. A., et al. 1995; Noll, A., et al., 1996; Pockley, A. G. and P. C. Montgomery, 1991) and IL-5 and -6 have been expressed from recombinant vaccinia virus vectors (Ramsay, A. J., et al., 1994; Ramsay, A. J., et al., 1993). In addition, IL-2, IL-8 and GM-CSF have been expressed from plasmid DNA (Hengge, U. R., et al., 1996; Xiang, Z. and H. C. Ertl, 1995; Chow, Y. H., et al., 1997). Our study is unique in its use of IL-6 DNA as a vaccine adjuvant administered by gene gun delivery. Interleukin-6 is a critical factor in end stage differentiation of B-cells into IgA secreting plasma cells (McGhee, J. R. and H. Kiyono, 1992; Holmgren, J., et al., 1992) and studies in IL-6 knockout mice demonstrated that IL-6 is vital for maintenance of mucosal IgA responses (Ramsay, A. J., et al., 1994). However, IL-6 also stimulates proliferation of T-cells (Van Snick, J., 1990). Immunity to influenza is similarly thought to be dependent upon both local IgA responses for protection at the mucosal surfaces and cellular immune responses for clearance of virus from the body (Murphy, B. R. and R. G. Webster, 1996). Our results demonstrate that administration of DNA encoding IL-6 as an adjuvant to HA DNA vaccination confers complete protection from pulmonary infection with influenza virus in mice.

B. Materials and Methods

Influenza virus and DNA vaccine preparation. A/Equine/Kentucky/1/81, a prototypical H3N8 equine influenza virus, was obtained from the influenza repository at the University of Wisconsin-Madison. The virus was propagated in the allantoic cavity of ten-day-old embryonated chicken eggs as previously described (Olsen, C. W., et al., 1993) and was not specifically adapted for replication in mice. The Eq/KY HA gene was cloned and sequenced previously (Olsen, C. W., et al., 1997). A cDNA encoding human IL-6 (huIL-6) was kindly provided by Dr. Robert Fenton (National Cancer Institute). Human IL-6 is known to bind to murine IL-6 (muIL-6) receptors and to be functional in the mouse (Van Snick, J., 1990). Its use in this study allowed us to distinguish between serum IL-6 activity expressed from our DNA construct versus endogenous muIL-6.

The cDNAs for both the Eq/KY HA and huIL-6 were cloned into a CMV promoter-based eukaryotic expression vector (pWRG, PowderJect®, Madison, Wis.) containing the intron A from CMV, the kanamycin resistance gene and a poly A signal (Olsen, C. W., et al., 1997). Hemagglutinin protein expression from the resulting plasmid was confirmed by immunofluorescent antibody staining of transiently transfected Madin-Darby canine kidney (MDCK) cells as previously described (Olsen, C. W., et al., 1997). Interleukin-6 expression was confirmed by testing the supernatant of transiently transfected MDCK cells using both a commercially-available ELISA kit (see below) and the B9 cell assay for IL-6 bioactivity (Aarden, L. A., et al., 1987). Plasmid DNA was prepared for gene gun administration by anion-exchange chromatography (Qiagen, Inc, Chatsworth, Calif.), adsorbed to gold beads and the beads coated into Tefzel plastic tubing as previously described (Olsen, C. W., et al., 1997; Haynes, J. R., et al., 1996). All DNA cloning procedures throughout this project were conducted using standard techniques (Ausubel, F. M., et al., 1989).

Vaccination protocols. DNA was administered using the PowderJect®XR gene delivery device. Two doses of 2.5 μg of DNA were administered into the epidermis of BALB/c mice, with a three week interval between vaccinations. The mice were divided into three vaccination groups. One group of mice (n=10 mice) received only pWRG plasmid DNA and served as controls. The second group of mice (n=20 mice) received HA+control pWRG DNA and the third group of mice received HA+IL-6 DNA (n=20 mice). The reason for including control pWRG DNA in the HA group was to equilibrate the amount of DNA given and to account for any promoter competition that may occur in the mice receiving HA+IL6 DNA. However, for the sake of clarity, this HA+pWRG combination is hereafter referred as HA DNA alone.

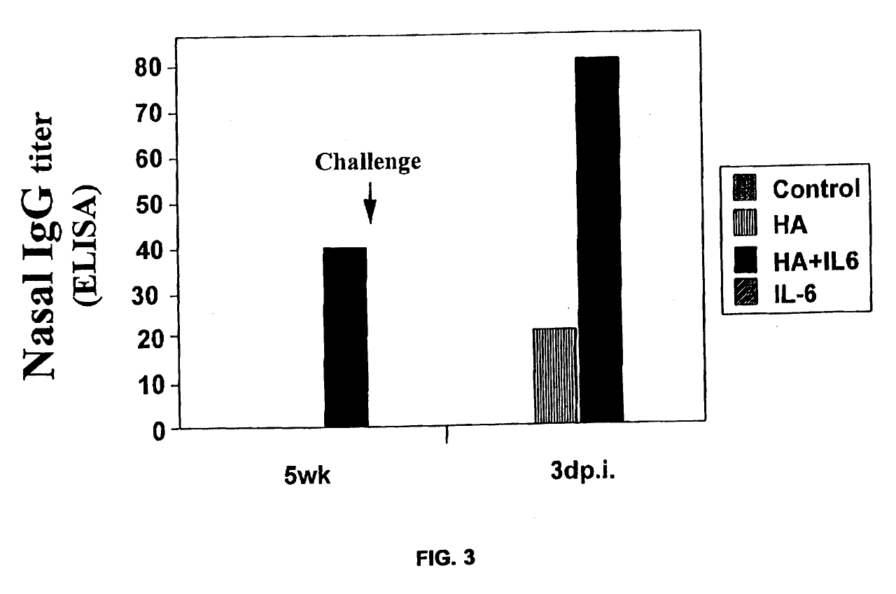

To confirm our initial protection from challenge results and to obtain samples for assessment of mucosal immune responses, a second round of similar experiments was conducted, this time including a fourth group of mice that received IL-6+pWRG DNA. This group was included as an additional control to rule out any direct protective effect of IL-6 in the absence of HA expression. Two weeks after the second vaccinations, all mice in the first experiment and half of the mice in each vaccination group in the second experiment were challenge-infected with 1×10^{7.4} egg infectious dose₅₀ (EID₅₀) units of Eq/KY virus by intranasal instillation under light Metofane (Pittman Moore, Mundelein, Ill.) sedation. The remaining mice in the second experiment were euthanized 2 weeks after their second vaccination to obtain nasal wash samples for assessment of mucosal immune responses (see below and FIG. 3) in the absence of a challenge infection. Challenged mice were euthanized either 3 or 5 days after infection in experiment 1. (Half of the mice in each group were euthanized on each day.) All challenged mice in experiment 2 were euthanized 3 days after infection.

Interleukin 6 assays. Levels of both huIL-6 and muIL-6 were determined on serum samples obtained 44 hours after the first and second vaccinations, using commercially-available ELISA kits (R&D Systems Inc., Minneapolis, Minn.). The assays were conducted as per the manufacturer's guidelines.

Samples for virus-specific Ab testing. Blood was collected from the supraorbital sinus for serologic testing immediately prior to the first vaccinations, immediately prior to the second vaccinations (3 weeks), immediately prior to challenge (5 weeks) and at the time of euthanasia. Blood samples from all of the mice in each vaccination group were pooled at the time of collection.

In the second experiment, nasal wash samples were collected to allow for assessment of local mucosal Ab responses in the upper airways. These samples were obtained 2 weeks after the second vaccination in mice that were not challenged and 3 days after infection in the challenged mice. Nasal washes were obtained by inserting a 22 gauge intravenous catheter retrograde from the tracheal bifurcation toward the head, positioning the end of the catheter at the caudal area of the nasal turbinates. One milliliter of sterile PBS+1% BSA was flushed through the catheter and collected as it drained from the nares into a sterile petri dish. The flush was repeated three times using the same volume of fluid.

As an additional measure of mucosal immune responses in the second experiment, virus-specific Abs were measured in fecal pellets that were collected immediately prior to challenge and at the time of euthanasia. One fecal pellet was collected per mouse and homogenized to a concentration of 1 mg/ml (W/V) in PBS containing 5% fetal bovine serum and 0.01% Tween 20.

ELISA and VN Ab assays. Virus-specific Abs were measured in serum, nasal washes and fecal suspensions using an isotype-specific ELISA assay as previously described (Olsen, C. W., et al., 1997). Appropriate positive and negative controls were included on each plate. The ELISA Ab titers were defined as the reciprocal of the highest dilution of sample for which the optical density (OD) was a least 2 times the OD of the negative control sample on that plate. The level of VN antibodies in serum was determined by inoculation (in triplicate) of Madin-Darby canine kidney cells with serial dilutions of serum incubated with 50 tissue culture infectious dose₅₀ (TCID₅₀)units of Eq/KY virus, as described previously (Olsen, C. W., et al., 1997). The VN Ab titers were calculated as the reciprocal of the highest dilution of serum that completely inhibited Eq/KY virus-induced cytopathic effect.

Virus titration. Lung tissue samples were homogenized in viral transport media using a Stomacher 80 lab blender (Tekmar Co., Cincinnati, Ohio). The level of infectious virus was determined by inoculation (in triplicate) of serial dilutions of each sample into the allantoic cavity of ten-day-old embryonated chicken eggs. After incubation at 35° C. for 72 hours, a sample of allantoic fluid from each egg was tested by hemagglutination assay (Palmer, D. F., et al., 1975) for the presence of virus. Virus titers were calculated in EID₅₀ units/g lung tissue by the method of Reed and Muench (Reed, L. J. and H. Muench, 1938). The titers of virus in the lungs of mice in each treatment group were compared statistically by one-way analysis of variance (ANOVA) and pairwise contrasts.

C. Results

Serum IL-6 levels. The levels of human and murine IL-6 were determined in serum samples obtained 44 hours after DNA vaccinations. Following the first vaccinations, the level of huIL-6 in serum was 40 pg/ml in the mice that received IL-6 DNA, but was below the level of detection (<3 pg/ml) in the mice that received control or HA DNA alone. Following the second vaccinations, huIL-6 levels were below the level of detection in all mice. In addition, muIL-6 levels remained below the level of detection (<15.6 pg/ml) in all samples, indicating that expression of huIL-6 by DNA administration did not up-regulate endogenous muIL-6 expression.

Virus-specific serum IgG, IgA and VN Ab responses.

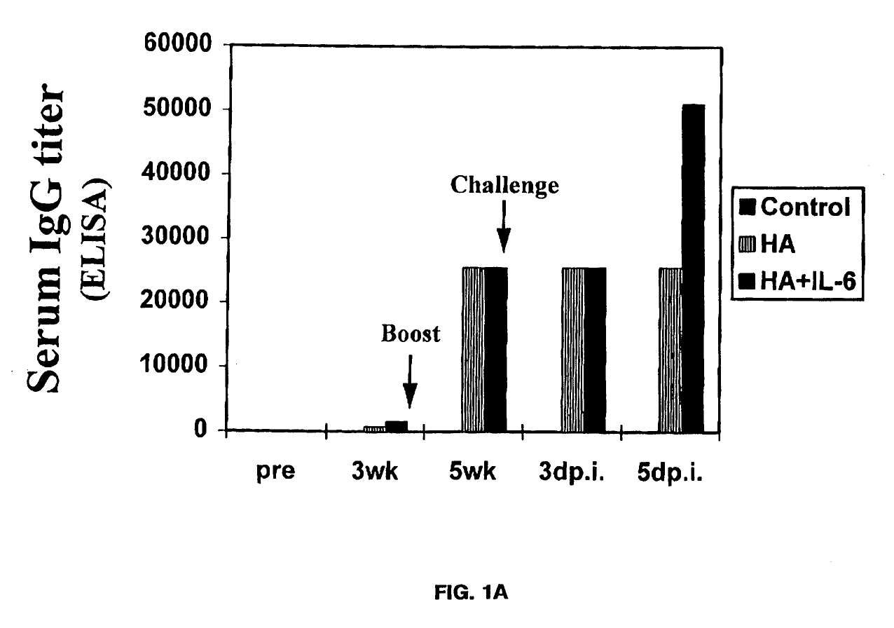

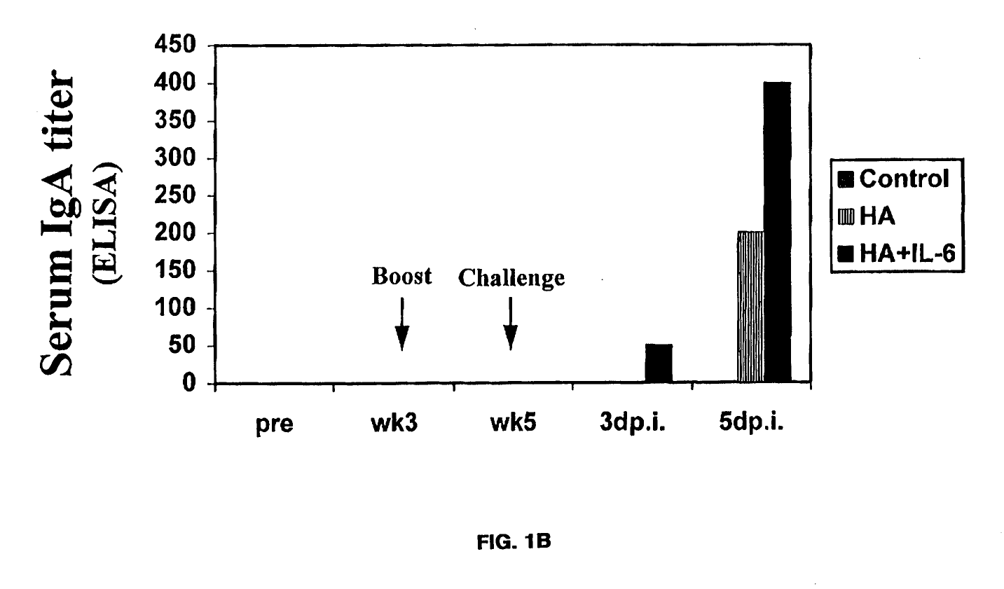

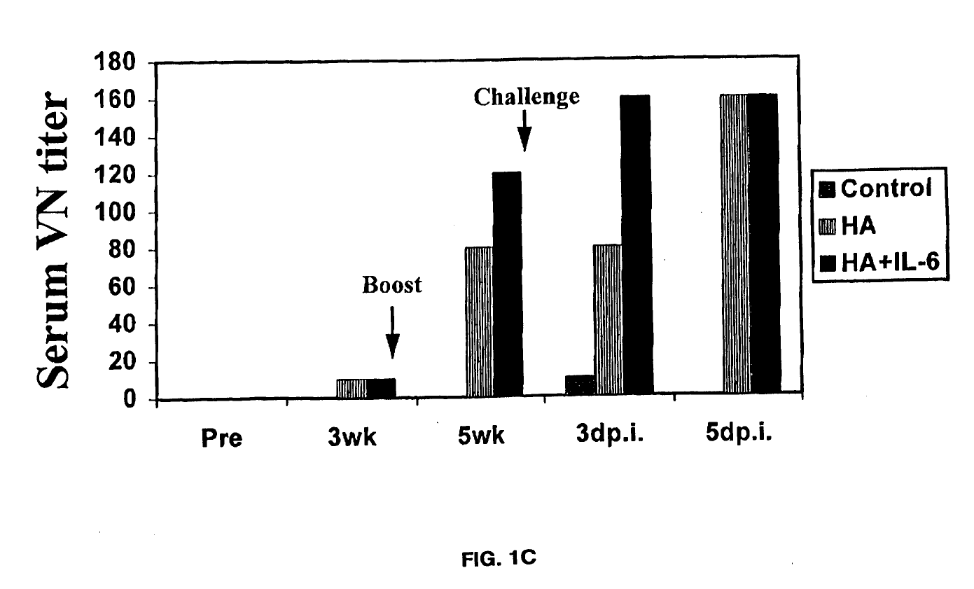

FIGS. 1(A) and (B) describe virus-specific serum IgG (A) and IgA (B), as measured by ELISA, in mice vaccinated with control pWRG DNA, Eq/KY HA DNA or Eq/KY HA DNA+ IL-6 DNA. The ELISA assays were performed as previously described (Olsen, C. W., et al., 1997). ELISA titers on the Y-axis are defined as the reciprocal of the highest dilution of serum for which the OD was a least 2 times the OD of the negative control sample on that plate. FIG. 1C describes virus-neutralizing Ab titers in mice vaccinated with control pWRG DNA, Eq/KY HA DNA or Eq/KY HA DNA+IL-6 DNA. Virus neutralizing Abs were measured by inoculation (in triplicate) of Madin-Darby canine kidney cells with serial dilutions of serum incubated with 50 TCID₅₀ units of Eq/KY virus. (See the Materials and Methods.) The VN titers on the Y-axis are defined as the reciprocal of the highest dilution of serum that completely inhibited Eq/KY virus-induced cytopathic effect.

The times of sampling are shown on the X-axis. The times of second vaccination (boost) and challenge infection are indicated by arrows.

Administration of HA and HA+IL-6 DNA induced Eq/Ky virus-specific serum IgG and VN Abs and primed the mice for production of virus-specific serum IgA following challenge. No antibodies to Eq/KY virus were detectable in the control mice that received pWRG control DNA or IL-6 DNA in the absence of HA DNA. Virus-specific IgG levels were negligible 3 weeks after the first vaccination, but rose to titers of 25,000 by 2 weeks after the second vaccination (immediately prior to challenge) in the mice that received either HA or HA+ IL-6 DNA (FIG. 1A). Virus-specific IgA was not detectable until after challenge infection in either group of mice. Following challenge, IgA appeared earlier in the mice that received HA+IL-6 DNA and rose to a higher titer (1 serum dilution) by 5 days after challenge, compared to the mice that were vaccinated with HA DNA alone (FIG. 1B).

The kinetics of the VN Ab responses paralleled those of the serum IgG responses. At the time of challenge, the VN titer of the mice that received HA+IL-6 DNA was 50% higher than the titer in the mice that received HA DNA alone.

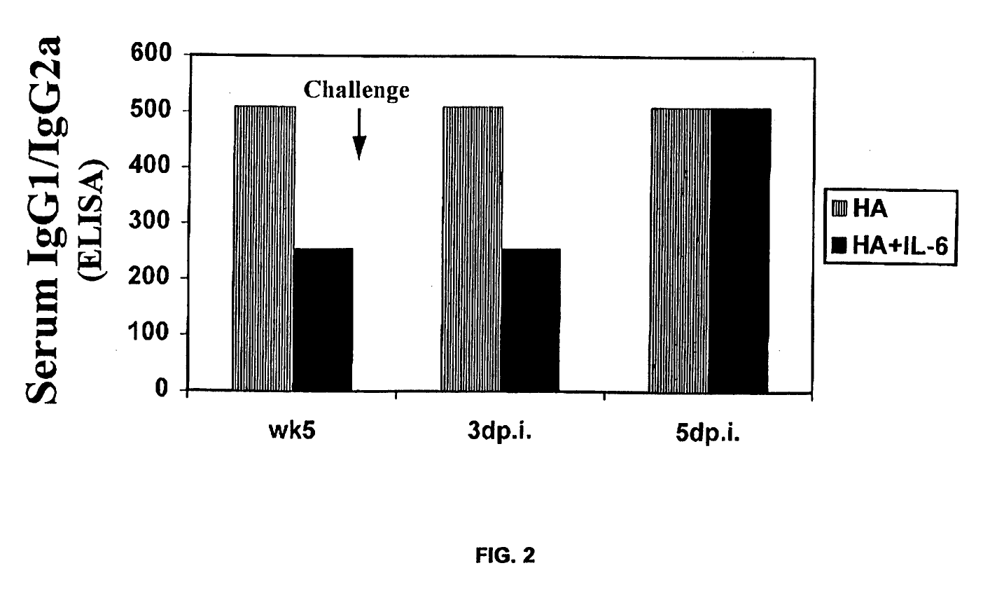

Virus-specific IgG subclass responses. In the mouse, the Th1 pathway favors the production of Abs of the IgG2a isotype and the Th2 pathway favors IgG1 production (Mosmann, T. R. and S. Sad, 1996; Finkelman, F. D., et al., 1990). We examined the ratio of IgG1 to IgG2a immediately prior to and following challenge as an indirect reflection of Th1/Th2 responses. FIG. 2 shows the ratio of virus-specific IgG1/IgG2a, as measured by ELISA, in the serum of mice vaccinated with control pWRG DNA, Eq/KY HA DNA or Eq/KY HA DNA+IL-6 DNA. The ELISA assays were performed using isotype-specific, horseradish peroxidase-conjugated, rabbit anti-mouse Ab as described previously (Olsen, C. W., et al., 1997). The IgG1 and IgG2a titers used to calculate the ratios shown on the Y-axis were defined as the reciprocal of the serum dilutions for which the ODs were a least 2 times the OD of the negative control sample on that plate. The times of sampling are shown on the X-axis. The times of second vaccination (boost) and challenge infection are indicated by arrows.

IgG1 responses predominated in all mice and the IgG1/2a ratios were largely similar in both the HA and HA+IL-6 DNA vaccinated mice. The only detectable difference was a 2-fold higher IgG1/2a ratio at the time of and 3 days after challenge in the mice vaccinated with HA DNA alone (FIG. 2).

Virus-specific mucosal IgG and IgA responses. In the second experiment, we assessed mucosal IgG and IgA responses to our DNA vaccination regimes. No virus-specific IgA was detectable in any of the nasal washes or fecal pellets tested. However, virus-specific IgG was detected in the nasal washes. In particular, virus-specific IgG was present in the nasal washes prior to challenge in the mice vaccinated with HA+IL-6 DNA, whereas it was not detectable until after challenge infection in the mice that received HA DNA alone. In addition, by 5 days after challenge, the virus-specific IgG titer was 4-fold higher in the mice vaccinated with HA+IL-6 DNA (FIG. 3).

FIG. 3 describes virus-specific IgG titers, as measured by ELISA, in nasal wash specimens from mice vaccinated with control pWRG DNA, Eq/KY HA DNA or Eq/KY HA DNA+IL-6 DNA. The ELISA assay was performed as described previously (Olsen, C. W., et al., 1997). Titers on the Y-axis are defined as the reciprocal of the highest dilution of sample for which the OD was a least 2 times the OD of the negative control sample on that plate. The times of sampling are shown on the X-axis. The times of second vaccination (boost) and challenge infection are indicated by arrows.

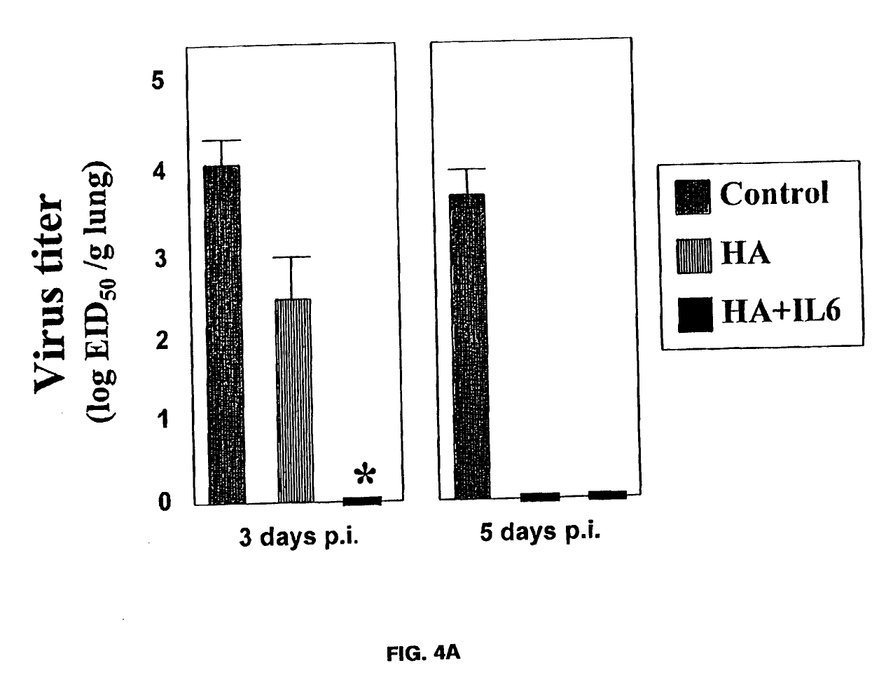

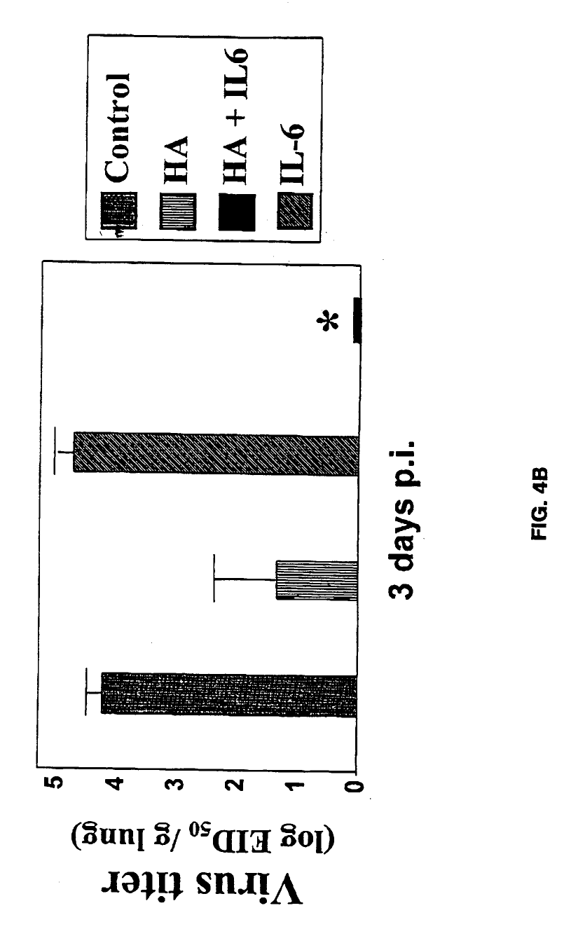

Protection from challenge infection. FIG. 4 shows the mean titers (and standard errors of the means) of virus in the lungs of mice following vaccination with control pWRG DNA, Eq/KY HA DNA , Eq/KY HA DNA+IL-6 DNA or IL-6 DNA alone. Mice were challenged with 1×10^{7.4} EID₅₀ units of Eq/KY virus by intranasal instillation under light Metofane (Pittman Moore) sedation, two weeks after the second vaccinations. FIG. 4(A) shows the results from experiment 1 and FIG. 4(B) shows the results from experiment 2. Virus was detected by inoculation (in triplicate) of serial dilutions of each of the mouse lungs into the allantoic cavity of ten-day-old embryonated chicken eggs (see the Materials and Methods). Mice were euthanized for collection of lung samples either 3 or 5 days after challenge, as shown on the X-axises.

In experiment 1 (FIG. 4A), compared to the control vaccinated mice, the mice that received HA DNA had reduced levels of virus in their lungs by day 3 after challenge and had cleared their infections by 5 days after challenge. However, the mice vaccinated with HA+IL-6 DNA were completely protected from pulmonary infection, as evidenced by a lack of virus in their lungs as early as 3 days after challenge. The difference in virus titers between the HA and HA+IL-6 DNA vaccinated mice 3 days after challenge is highly statistically significant (p<0.0001). These results were confirmed in a second experiment that also included a group of mice that were given IL-6 DNA alone (FIG. 4B). Once again, the mice that received both HA+IL-6 DNA were completely protected. The virus titers in the mice that received IL-6 DNA without HA DNA were comparable to those in the control DNA vaccinated mice, thus confirming that the protection we observed cannot be attributed to any effect of IL-6 in the absence of co-administered specific antigen.

D. Discussion

Infection with equine influenza virus is the most common respiratory illness of horses in North America (Mumford, J., 1992; Traub-Dargatz, J. L., et al., 1991). Unfortunately, commercially-available inactivated virus vaccines offer only very limited, short-term protection, (Traub-Dargatz, J. L., et al., 1991) although recovery of horses from natural infection leads to long-lasting protection (Hannant, D., et al., 1988). Likewise, we have previously demonstrated that recovery from Eq/KY infection in BALB/c mice confers complete immunity to subsequent challenge infection (Olsen, C. W., et al., 1997). However, in those initial studies, Eq/KY HA DNA vaccination could not induce a similar level of complete protection unless a very prolonged time period was provided between doses of vaccine (Olsen, C. W., et al., 1997). In the present study, we have investigated whether co-administration of DNA encoding the huIL-6 gene would enhance the efficacy of HA DNA vaccination. Our results clearly indicate that gene gun co-administration of HA and IL-6 DNA confers much greater protection from pulmonary infection with Eq/KY virus in mice than does administration of HA DNA alone. The mice that received HA+IL-6 DNA were completely protected from pulmonary infection, with no detectable virus in their lungs after challenge (FIGS. 4A and 4B).

A number of cytokines have been investigated previously as vaccine adjuvants, including IL-1, -2, -3, -4, -5, -6, -7, -8 and -12, IFN-gamma, GM-CSF and TNF-alpha (Lin, R., et al., 1995; Lofthouse, S. A., et al., 1995; Noll, A. and I. B. Autenrieth, 1996; Pockley, A. G. and P. C. Montgomery, 1991; Ramsay, A. J., et al., 1994; Ramsay, A. J., et al., 1993; Hengge, U. R., et al., 1996, Xiang, Z. and H. C. Ertl, 1995; Chow, Y. H., et al., 1997; Geissler, M., et al., 1997). We specifically chose to use IL-6 because of its pleiotropic effects on both the humoral and cellular components of the immune system. Interleukin-6 functions as a co-stimulatory molecule for T-cell proliferation (Van Snick, J., 1990) and it enhances B cell differentiation and IgA production (McGhee, J. R. and H. Kiyono, 1992; Holmgren, J., et al., 1992). In particular, IL-6 has been shown previously to have an adjuvant effect on IgA responses in tears (Pockley, A. G. and P. C. Montgomery, 1991) and Ramsay, et al. found that humoral immune responses in the respiratory tract to vaccinia virus-expressed influenza hemagglutinin were reduced in IL-6 knock-out mice (Ramsay, A. J., et al., 1994). In addition, IL-6 over-expression results in enhanced serum IgA responses (Brandt, S. J., et al., 1990).

Along with the dramatic enhancement of protection from challenge that we observed in the mice that received HA+IL-6 DNA, we also observed differences in their immune responses compared to the mice that received HA DNA alone. Virus-specific serum IgA appeared sooner and IgG and IgA titers were 2-fold higher in the mice that received HA+IL-6 DNA.

In addition to assessing total virus-specific serum Ab responses by ELISA, we also measured the level of VN Abs in the serum. The mice that received HA+IL-6 DNA had a higher VN titer (120 vs. 80) at the time of challenge.

One intriguing immunologic finding in our study was the presence of virus-specific IgG Ab in nasal washes. This was the immunologic parameter that differed most substantially at the time of challenge between the mice that received HA+IL-6 DNA and those that received HA DNA alone. Virus-specific nasal IgG was present at a titer of 40 immediately prior to challenge in the HA+ IL-6 DNA mice, whereas nasal IgG was not present in the mice vaccinated with HA DNA alone until after challenge, and then only to a titer of 20. The procedure for obtaining nasal wash samples is very non-traumatic and we did not encounter bleeding during the collections. Therefore, we are confident that this IgG does not simply represent blood contamination at the time of sampling. It is also interesting that virus-specific nasal and fecal IgA remained below the level of detection in all of the mice, even after challenge. Immunoglobulin A is the predominant isotype in mucosal secretions because it is selectively transported across mucosal surfaces. Immunoglobulin G cannot utilize this same polymeric immunoglobulin receptor transport system and, therefore, most likely reaches the mucosal secretions either via transudation from systemic sources or by an alternative mechanism following local production. Results from several studies indicate that protection from initial infection in the upper airway of mice (Bender, B. S., et al., 1996; Liew, F. Y., et al., 1984; Renegar, K. B and P. A. Small, Jr., 1991; Renegar, K. B and P. A. Small, Jr., 1991; Tamura, S., et al., 1991; Takase, H., et al., 1996) and humans (Murphy, B. R. and R. G. Webster, 1996; Clements, M. L., et al., 1983) correlates with local IgA responses. However, there is precedent for protection from influenza virus lethal challenge in mice in the absence of a mucosal IgA response (Deck, R. R., et al., 1997). To more completely understand the immunologic basis for the IL-6 adjuvant effect, future studies will determine whether mice that receive HA+IL-6 DNA are protected from initial infection in the nasal passages, as well as in the lungs, and the role of cell-mediated immune responses.

In summary, gene gun administration of DNA encoding IL-6 induced a very significant adjuvant effect on the protection from challenge infection elicited by an equine influenza virus HA DNA vaccine in mice. We are currently initiating similar studies in horses themselves, as well as in pigs, where influenza virus is also an important pathogen. Immune responses against influenza virus have been demonstrated previously using gene gun-mediated DNA vaccination in both of these species (Swain, et al., unpublished results; Lunn, et al., unpublished results), but as in our mouse model system, HA DNA vaccination alone has not been sufficient to induce complete protection from challenge. Therefore, we have cloned the porcine IL-6 gene into our vaccine expression vector and demonstrated that it is functionally expressed in vitro (Larsen, et al., unpublished results) and have similarly prepared an equine IL-6 clone (see below) to allow us to assess the adjuvant effect of IL-6 on DNA vaccination in these species.

2. Co-administration of Plasmid DNA Encoding Human IL-6 with a Swine Influenza Virus Hemagglutinin DNA Vaccine in Mice

A. In General

We initially demonstrated (as described above) that co-administration of DNA encoding human interleukin-6 (huIL-6) enhances immune responses to an equine influenza virus hemagglutinin DNA vaccine and subsequent protection from homologous virus challenge infection in mice with A/Equine/Kentucky/1/81, an H3N8-subtype influenza virus. In this section we demonstrate that co-administration of huIL-6 DNA also enhances protective immunity generated by DNA vaccination against a different influenza virus, an H1N1-subtype swine influenza virus.

Infection of pigs with H1N1 influenza A virus is an important problem for the swine industry throughout the world. Influenza virus infection in pigs can occur as an enzootic problem in a herd, or as explosive outbreaks of acute respiratory disease with fever, anorexia, lethargy, weight loss, nasal and ocular discharge, coughing and fulminant dyspnea. Although rarely fatal, swine influenza can be of substantial economic impact because of costs for veterinary care and a delay in reaching market weight (Easterday, B. C. and V. S. Hinshaw, 1992).

Beyond the impact of influenza for the swine industry, pigs are also very important in the ecology and evolution of influenza A viruses in humans. The major pandemics of human influenza this century were caused by strains that were reassortants between pre-existing human and avian viruses (Webster, R. G., et al., 1992). Because the respiratory tract of pigs contains receptors for both avian and mammalian influenza viruses (Ito, T. and Y. Kawaoka, 1998), pigs are uniquely susceptible to infection with viruses of avian (Guan, Y., et al., 1996; Kida, H., et al., 1994; Pensaert, M., et al., 1981; Scholtissek, C., et al., 1983), as well as swine and human origin (Nakajima, K., et al., 1982; Ottis, K., et al., 1982; Shu, L. L., et al., 1994; Zhou, N., et al., 1996). As such, they have been implicated as intermediate species for adaptation of avian viruses to mammals (Campitelli, L., et al., 1997) and as the “mixing vessels” in which human-avian virus reassortment occurs (Scholtissek, C., et al., 1983; Scholtissek, C. and E. Naylor, 1988; Webster, R. G., et al., 1992). Most recently, human-avian virus reassortants have been isolated from commercially-raised pigs in Europe (Castrucci, M. R., et al., 1993), and subsequently, from children in The Netherlands (Claas, E. C. J., et al., 1994). The later point emphasizes the fact that pigs also pose a threat as direct sources for zoonotic transmission of swine influenza viruses (Dasco, C. C., et al., 1984; de Jong, J. C., et al., 1988; Hinshaw, V. S., et al., 1978; Wentworth, D. E., et al., 1997), sometimes resulting in the death of the people infected (Eason, R. J. and M. D. Sage, 1980; Kimura, K., et al., 1998; Patriarca, P. A., et al., 1984; Rota, P. A., et al., 1989; Top, F. H. and P. K. Russell, 1977; Wentworth, D., et al., 1994).

One potential mechanism for control of influenza virus infection in pigs is vaccination. Only one inactivated whole virus vaccine is commercially available in the U.S. Although this vaccine can reduce signs of illness and virus shedding, it does not provide the complete protection from infection and clinical disease that occurs following recovery from natural infection (Brown, G. B. and J. K. McMillen, 1994; Macklin, M. D., et al., 1998). We have, therefore, investigated DNA vaccination as an alternative approach.

B. Materials and Methods

Influenza virus and DNA vaccine preparation. A/Swine/Indiana/18726/88 (H1N1) (Sw/IN), a prototypical swine influenza virus, was obtained from the influenza virus repository at the University of Wisconsin-Madison. This virus was propagated in embryonated chicken eggs as described previously in this application for our equine influenza virus experiments. The HA gene of Sw/IN was amplified by reverse transcriptase-polymerase chain reaction and cloned into pUC18 plasmid (Noble, S., et al., 1993). Thereafter, the HA gene was subcloned into a eukaryotic expression plasmid containing the promoter and intron A of human CMV and the ampicillin resistance gene, creating a swine influenza virus HA DNA vaccine plasmid hereafter referred to as pWRG1683 (Macklin, M. D., et al., 1998). This plasmid is very similar to the equine HA DNA vaccine plasmid described previously in this application, except that it contains the H1 swine influenza virus HA, gene instead of the H3 equine influenza virus HA gene, and it contains ampicillin antibiotic resistance instead of kanamycin. The plasmid expressing huIL-6 used in these experiments is that same as that used in our equine influenza virus experiments. All DNA and gold bead preparation for gene gun administration was conducted as described previously in this application.

Vaccination protocol. Three groups of BALB/c mice were established. DNA vaccines were administered by Accell gene gun as described previously in this application. Each mouse was vaccinated twice, on days 0 and 21. One group of mice (n=16) received only 5.0 μg pWRG plasmid DNA and served as controls. The second group of mice (n=16) received 2.5 μg pWRG1683+2.5 μg pWRG (HA DNA vaccinates), and the third group of mice (n=16) received 2.5 μg pWRG1683+2.5 μg pWRGhuIL6 (HA+ IL-6 vaccinates). Two weeks following the second vaccination, 8 mice in each group were euthanized for collection of serum, cervical and mediastinal lymph nodes (CLN and MLN) and spleen samples for immunological assays. The remaining mice were challenge-infected by intranasal instillation of 10^{3.4} EID₅₀ of Sw/IN. (Note: because Sw/IN is 140-fold more highly infectious for mice than Eq/KY virus (Larsen, D. L., et al., 1998), this amount of virus is approximately equivalent in mouse infectious dose₁₀₀ units [MID₁₀₀] to the 10^{7.4} EID₅₀ challenge dose of Eq/KY used in our earlier experiments (27.7 MID₁₀₀ of Eq/KY versus 50 MID₁₀₀ of Sw/IN). Three days following challenge, the mice were euthanized, the amount of infectious virus in their lungs was determined, and serum and tissue samples as listed above were collected for immunological assays.

Virological and immunological assays. Virus titrations by growth in eggs and detection of virus-specific antibodies (Abs) in serum by ELISA were conducted as described previously in this application, with the exception that Sw/IN was used as the target Ag in ELISA. As an additional measure of immune responses in this experiment, antibody-secreting cells (ASC) were enumerated using an enzyme-linked immunosorbent spot (ELISPOT) assay. This assay allows one to detect the presence of virus-specific B lymphocytes secreting Ab of the IgG or IgA isotypes in specific locations in the body. We enumerated ASC in CLN, MLN and spleen. Single cell suspensions from these organs are prepared by tissue disruption through a stainless steel mesh screen, after which the red blood cells are lysed with ammonium chloride and viable cells counted using trypan blue dye. The cell preparations are then assayed in 96 well plates on nitrocellulose membranes coated with either 100 HA units of Sw/IN as specific Ag or 100 HA units of an antigenically unrelated influenza B virus as a negative control. After blocking the membranes with 5% fetal bovine serum (FBS), cells are added at concentrations of 10³ to 10⁶ cells/well and incubated on the Ag-coated membranes overnight at 37° C. In this assay, Abs that virus-specific lymphocytes secrete during incubation bind to the underlying Ag in a discrete “spot.” Following washing to remove the cells, these spots are detected by incubation with alkaline phosphatase-conjugated anti-mouse IgG or IgA and NTB/BCIP color reagent. The numbers of virus-specific ASC are reported as number of spots/10⁶ starting cells.

C. Results and Discussion

Consistent with the results of our previous equine influenza virus study, co-administration of huIL-6 DNA and swine influenza virus HA DNA enhanced protection from challenge infection and virus-specific immune responses in mice compared to administration of HA DNA alone.

Protection from challenge infection. HA DNA vaccination alone lead to only a slight reduction in virus titers in the lungs compared to controls, whereas HA+IL-6 DNA vaccination induced a highly statistically significant level of protection (p=0.004, ANOVA analysis), including 4/8 mice that were completely protected and had no detectable virus in their lungs after challenge. The mean virus titers in the lungs of mice in this experiment (± standard errors of the means [SEM]) are shown in the table below.

TABLE 1

| | Mean virus titer (log EID₅₀/g |
|---|---|
| | lung tissue) ± SEM 3 days after |
| | challenge infection with 10^{3.4} |
| Vaccination Group | EID₅₀ of Sw/IN |
| Control (pWRG alone) | 6.68 (0.16) |
| HA DNA (pWRG1683 + | 6.14 (0.43) |
| pWRG) | |
| HA + IL-6 DNA (pWRG1683 + | 2.46 (1.0)* |
| pWRGhulL6) | |

*statistically significant enhancement of protection compared to HA DNA alone (p = 0.004, ANOVA)

We observed the following immunologic responses:

(i) Virus-specific serum IgG titers immediately prior to challenge were 2-fold higher in the HA+IL-6 vaccinated mice than the mice that received HA DNA (pWRG1683) alone.

(ii) The numbers of virus-specific IgG ASC in the spleens of the HA+IL-6 vaccinated mice were 4-fold higher at 2 weeks after the second vaccination compared to the mice vaccinated with HA DNA alone (2.1 vs. 0.6 spots/10⁶ cells)

(iii) The HA+IL-6 DNA vaccinated mice had detectable virus-specific IgA ASC in their spleen prior to challenge (1.5 spots/10⁶ cells), whereas the HA DNA alone mice did not.

(iv) The HA+IL-6 vaccinated mice were primed for a virus-specific ASC response in the MLN after challenge (4.8 spots/10⁶ cells), whereas the mice receiving only HA DNA had no detectable virus-specific ASC in their MLN. This ability of the HA and IL-6 DNA co-administration regime to induce virus-specific ASC in the MLN is particularly significant. We (Larsen, D. L. and C. W. Olsen, 1998) and others (Justewicz, D. M., et al., 1995) have previously shown that the MLN is an important site of ASC responses following experimental influenza virus infection in mice, and it is clear that mice that have recovered from such infections are solidly immune to subsequent virus challenge (Larsen, D. L. and C. W. Olsen, 1998). In addition, preliminary results indicate that MLN are also a prominent site of virus-specific primary and memory B cell responses following experimental Sw/IN infection in pigs (Larsen, D. L. and C. W. Olsen, 1998). Therefore, we are confident that co-administration of Sw/IN HA and porcine IL-6 DNA will also lead to protection of pigs from challenge infection.

3. Co-administration of HA and huIL-6 DNA Enhances Protection Following High-dose Sw/IN Challenge Infection in Mice

A. Introduction

As a follow-up to the results outlined in Experiment 2, we conducted a second study to examine whether co-administration of HA and huIL-6 DNA would enhance protection from infection with a dramatically higher dose of challenge infection.

B. Materials and Methods

Three groups of BALB/c mice were established (n=10 mice/group). The mice were vaccinated on days 0 and 21 with a total of 5.0 μg of DNA, either pWRG alone (controls), pWRG1683+pWRG (HA vaccinates) or pWRG1683+ pWRGhuIL6, and then challenged 2 weeks after the second vaccination with 10^{6.2} EID₅₀ Sw/IN. (This represents an approximately 3-log increase in challenge level compared to Experiment I, and, based upon prior infectivity results (Larsen, D. L., et al., 1998) is equal to 31,623 MID₁₀₀.

C. Results and Discussion

With this extremely high dose of challenge virus, all of the mice, regardless of vaccination, became infected and replicated virus in their lungs. Remarkably however, co-administration of HA+IL-6 DNA still enhanced the level of protection compared to HA DNA vaccination alone. The lungs of the HA+IL-6 DNA vaccinated mice contained a significantly (p=0.04, ANOVA) lower amount of virus after challenge compared to the HA DNA alone vaccinates. The mean virus titers in the lungs ± standard errors of the means [SEM] are shown in the table below.

TABLE 2

| | Mean virus titer (log EID₅₀/g |
|---|---|
| | lung tissue) ± SEM 3 days after |
| | challenge infection with 10^{7.4} |
| Vaccination Group | EID₅₀ of Sw/IN |
| Control (pWRG alone) | 6.66 (0.32) |
| HA DNA (pWRG 1683 + | 4.86 (0.42) |
| pWRG) | |
| HA + IL-6 DNA (pWRG1683 + | 3.33 (0.72)* |
| pWRGhulL6) | |

* statistically significant enhancement of protection compared to HA DUNA alone (p = 0.04, ANOVA)

These data indicate that co-administration of IL-6 and HA DNA represents a powerful approach to vaccination that offers benefits over HA DNA vaccination alone even in the face of an extreme challenge dose of virus.

4. Use of Equine Il-6 DNA as an Adjuvant to DNA Vaccination

A. Introduction

Prevention of many infectious diseases is critically dependent on vaccination. Successful vaccines frequently mimic natural infection, especially when the disease naturally induces long lasting protective immunity. A good example is equine influenza virus, in which infection induces protective immunity for over 12 months and is associated with nasal IgA, and serum IgGa and IgGb antibody isotype responses. In sharp contrast current vaccines provide incomplete, short lived protection. Therefore, we investigated DNA vaccination of horses with the equine influenza virus hemagglutinin gene. Our results showed complete clinical protection and partial virological protection that were associated with IgGa and IgGb antibody responses in the absence of a mucosal IgA response. Despite the success of this initial DNA vaccination approach, improvement of the strategy remains important. Specifically the complete prevention of viral shedding in clinically protected horses could significantly decrease the viral transmission rate in the horse population. A second generation of DNA vaccines that uses the co-administration of cytokine genes as adjuvants has the potential to enhance immune responses to DNA vaccination alone. Our efforts are focused on interleukin 6, a cytokine with a wide range of activities in acute phase reactions and the regulation of B and T cell functions. In particular IL-6 regulates isotype switching and promotes T-Helper-2 responses, which have a pivotal role in mucosal immunity.

We, therefore, proposed the hypothesis that co-administration of an equine IL-6 plasmid will increase the protective immunity resulting from DNA vaccination of horses.

B. Subcloning and expression of equine IL-6 for gene gun vaccination

Equine interleukin 6 has previously been cloned and sequenced, and was kindly provided to us by Dr. David Horohov, Louisiana State University, Baton Rouge. In order to express IL-6 in a mammalian system, it was necessary to subclone the insert out of its original vector (pCREQIL6) into the gene gun expression vector (pWRG1647) that was provided by PowderJect Vaccines. The IL-6 insert was cut out of its original vector at the HindIII and NotI site using HindIII and NotI restriction enzymes and ligated into the pWRG1647 vector at the same sites using T4 DNA ligase.

The newly synthesized ligation product was then transformed into E. coli DHL5 and grown overnight in the presence of ampicillin. Colonies were picked the following day and the plasmid DNA was purified using anion exchange resin chromatography (Qiagen). Clones containing inserts of the correct size were identified by a test digest and visualized on an agarose gel. Taq dye terminator cycle sequencing was performed to verify the correct orientation of the insert and exclude clones containing mutations.

In order to test eqIL-6 protein expression in mammalian cells, the plasmid DNA (pWRGeqIL6) was grown up in larger amounts in E. coli and purified by anion exchange resin chromatography. Subsequently the plasmid DNA was coated onto gold beads and transfected into CHO cells using the Powderject® gene delivery device, a biolistic microparticle delivery device. CHO cell supernatants were collected 24 and 48 hours post transfection and stored at −20° C. until further examination. As positive control we collected CHO cell supernatants from cells transfected with human IL-6 and as a negative control we collected supernatants from CHO cells expressing empty plasmid (pWRG 1647).

The IL-6 activity of the CHO cell supernatants was tested using the B9 murine cell line, the viability of which depends on the presence of IL-6. Assays are performed by incubating B9 cells in the presence of test supernatants for 72 hours, and then measuring cell viability.

To confirm the specificity of equine IL-6 for the B9 cell IL-6 receptor, we also performed experiments in which we blocked binding by incubation with an anti-IL-6 receptor monoclonal antibody, prior to the addition of test supernatants. In all experiments we included a positive standard by adding known concentrations of recombinant human IL-6 to the control wells and a negative standard which were B9 cells in media alone. The viability of the B-9 cells was measured by addition of the tetrazolium salt, XTT. Live cells convert XTT to a colored form, which is detectable with a spectrophotometer at 450 nm.

Empty vector supernatants, independent of treatment, did not support B9 cell viability. High B9 cell viability confirms the successful expression of human IL-6 and activity was blocked by prior incubation of B9 cells with anti-IL-6 receptor antibody. The same pattern was seen with supernatants from equine IL-6 transfected CHO cells, and similarly B9 cell viability was reduced by the antibody to the IL-6 receptor. These experiments confirmed the functional activity of the pWRGeqIL-6 vaccine plasmid in mammalian cells.

C. Adjuvant Activity of Equine IL-6 DNA in DNA Vaccination of Horses with the Equine Influenza Virus Hemagglutinin (HA) Gene

An experiment was carried out using two groups of seven influenza virus naive ponies, which were maintained in isolation suites for the duration of the experiment. Both groups of ponies were subjected to DNA vaccination using the same plasmid expressing the equine influenza virus HA gene that was used in the mouse experiments described previously in this application. Horses will be vaccinated at skin and mucosal (tongue and conjunctal surface of the eye) sites using protocols previously demonstrated by us to be effective in generating protective anti-influenza virus IgGa and IgGb antibody responses.

In one of these vaccination groups the HA DNA was combined with equine IL-6 DNA. In the other group the HA DNA was combined with a similar amount of DNA consisting of empty vector as a control. Three vaccinations were administer at approximately 60 day intervals, and 14 days after the third vaccination three ponies from each vaccination group were euthanized for measurement of influenza virus specific lymphoproliferative responses in regional lymphoid tissues. At the same time lymphoproliferative responses were measured in the same tissues in four influenza virus naive ponies, and four further ponies that had recovered from an influenza virus infection fourteen days previously.

A total of 45 days after the third vaccination the remaining 4 ponies in each DNA vaccination group were subjected to an intranasal influenza virus challenge; an identical challenge was administered to three seronegative control ponies.

All seven naive control ponies that were subjected to an influenza virus challenge showed clinical signs of infection, including fever, coughing, and mucopurulent nasal discharge. In addition all seven ponies shed virus for up to seven days post-challenge. None of the four ponies in either DNA vaccination group showed any clinical signs of disease after challenge infection. In each DNA vaccination group, only one pony shed virus in excess of two days post-viral challenge, and in each instance at lower titers than in the control ponies. Both DNA vaccination pony groups generated similar strong influenza virus specific serum antibody responses of the IgGa and IgGb antibody isotypes subsequent to the second and third DNA vaccinations; no other antibody isotype responses were detected prior to challenge infection.

Subsequent to challenge infection the two DNA vaccination groups could be clearly distinguished in that the equine IL-6 DNA adjuvanted group developed a strong IgG(T) isotypic response far in excess of that generated by HA DNA vaccination alone. In addition the equine IL-6 DNA adjuvanted group developed a stronger respiratory tract mucosal IgA response than that generated by HA DNA vaccination along. This demonstrates that use of equine IL-6 DNA as a an adjuvant results in stronger priming of the equine immune response as evidenced by induction of an additional serum IgG sub-isotypic response, and increased mucosal IgA responses in response to challenge infection.

In lymphoproliferative responses a similar augmentation of influenza virus specific immune responses by use of equine IL-6 DNA. This was seen in the regional lymph nodes in the inguinal area which drain skin regions used for administration of DNA vaccination. After preparation of lymphocytes from these tissues and stimulation with influenza virus, both DNA vaccination groups showed greater proliferative responses than either naive control ponies or ponies previously infected with influenza virus. However, this responses were more than two standard deviations higher in the ponies administered the equine IL-6 DNA as a an adjuvant.

Overall, the use of equine IL-6 DNA shows strong evidence of being able to both amplify and modify influenza virus-specific immune responses in the horse, and, consequently, may be of considerable value in improving protective immunity resulting from DNA vaccination in this species.

5. Co-deliver of DNA Expressing hepsAg (7128) and DNA Expressing Human IL-6 in Mice

A. In General

In the following experiment we demonstrate that co-delivery of DNA encoding the human hepatitis surface antigen (SAG) a DNA expressing human IL-6 provoked an enhanced CTL response in the mouse model system. These results demonstrate that the DNA vaccination/IL-6 co-administration method may have usefulness in broader applications, including cancer vaccines.

B. Materials and Methods

Expression vectors: The expression vector WRG7128 encodes human hepatitis surface antigen (sAg). WRG7077 is the empty control vector missing the insert sequences encoding sAg. The expression vector encoding human IL-6 (hIL-6) was obtained as above.

Immunizations: Balb/C female mice (8-10 weeks) were immunized with the PowderJect® XR gene delivery device at weeks 0 and 4. Each mouse received two targets consisting of a co-delivery of two expression vectors into the upper and underside of the tongue or abdominal skin as follows:

Group 1: 1.25 μg sAg (7128)+1.25 μg hIL-6 delivered to the tongue.

Group 2: 1.25 μg sAg (7128)+1.25 μg (7077) delivered to the tongue.

Group 3: 1.25 μg sAg (7128)+1.25 μg hIL-6 delivered to the skin.

Group 4: 1.25 μg sAg (7128)+1.25 μg (7077) delivered to the skin.

Group 5 (Negative Controls): 1.25 μg (7077)+ 1.25 μg (hIL-6) delivered to either the skin or tongue.

CTL Assay: A chromium release assay was used to measure the ability of in vitro-stimulated splenocytes to lyse target cells expressing hepatitis surface antigen. Mice were sacrificed 2 weeks following the final immunization. Splenocytes were harvested and red blood cells lysed with ACK lysis buffer (Sigma). Following three washes with RMPI-10 (RPMI-1640 plus 10% heat-activated fetal calf serum and 50 μg/ml gentamycin), splenocytes were resuspended to 6×10⁶ cells per ml in SM medium (RPMI-10 plus 1 mM sodium pyruvate plus 1x nonessential amino acids). Stimulator cells were mitomycin C-treated splenocytes from naive mice pulsed with a synthetic peptide representing a known hepsAg CTL epitope (IPQSLDSWWTS). A total of 2.0×10⁶ stimulator cells were cocultivated with 6×10⁶ responder cells from immunized mice in a 24-well culture plate in SM supplemented with 10 U of rat IL-2/ml (Collaborative) for 6 days at 37° C. and for 5 hours with 30,000 radiolabelled target cells (P815 cells transformed with a vector expressing hepatitis surface antigen) at various effector/target ratios. Target cell lysis was measured by liquid scintillation counting of 40 μl of cell supernatants. Percent-specific lysis of labeled target cells for a given effector cell sample was calculated as follows:

$$\% Specific lysis = 100 \% \times \frac{( Cr51 release in sample - Spontaneous Cr51 release )}{( Maximum Cr51 release - Spontaneous Cr51 release )}$$

Spontaneous chromium release represents the amount of radioactivity released from target cells in the absence of effector cells. Maximum chromium release represents the amount of radioactivity released following lysis of target cells with 1% Triton X-100. No specific lysis above background levels was observed for the experimental or control effector cells against P815 cells not expressing hepatitis surface antigen (non-specific lysis).

C. Results

Table 3, below, demonstrates an increased CTL response with mice immunized with a co-delivery of IL-6 versus mice immunized with a co-delivery including an empty vector.

Percent specific lysis at an effector:target ratio of 50/1 was compared in mice immunized with a codelivery of WRG7128+IL-6 versus mice immunized with a codelivery of WRG7128+an empty vector (WRG7077). Mice were immunized in the skin or tongue as indicated. A statistical difference between these groups was determined by paired T-test analysis (p<0.05).

TABLE 3
Effector: Target Ratio = 50/1

| Target | WRG7128 + IL-6 | WRG7128 + WRG7077 |
|---|---|---|
| Site | (% specific lysis-control lysis) | (% specific lysis-control lysis) |
| Skin | 49.8 | 19.7 |
| Skin | 48.5 | 32.5 |
| Skin | 27.2 | 34.0 |
| Tongue | 24.9 | −7.7 |
| Tongue | 30.5 | 14.0 |
| Tongue | 19.2 | 1.2 |

To confirm these results, percent specific lysis was also compared between these two groups at an effector:target ration of 17/1. At this E:T, the difference between mice receiving IL-6 versus the empty vector was also statistically significant (p<0.01).

TABLE 4
Effector: Target Ratio = 17/1

| Target | WRG7128 + 11-6 | WRG7128 + WRG7077 |
|---|---|---|
| Site | (% specific lysis-control lysis) | (% specific lysis-control lysis) |
| Skin | 40.5 | 9.8 |
| Skin | 43.1 | 23.0 |
| Skin | 20.2 | 18.2 |
| Tongue | 17.9 | −3.5 |
| Tongue | 25.9 | 11.5 |
| Tongue | 15.1 | 1.0 |

### REFERENCES

Aarden, L. A., De Groot, E. R., Schapp, O. L., Lansdorp, P. M., “Production of hybridoma growth factor by monocytes,” Eur. J. Immunol. 17:1411-6, 1987.

Anderson, R., Gao, X. M., Papakonstantinopoulou, A., Roberts, M., Dougan, G., “Immune response in mice following immunization with DNA encoding fragment C of tetanus toxin,” Infect. Immun. 64:3168-73, 1996.

Ausubel, F. M., Brent, R., Kingston, R. E., Moore, D. D., Seidman, J. G., Smith, J. A., Struhl, K., “Current protocols in molecular biology,” New York: Greene Publishing Associates and Wiley-Interscience, 1989.

Bender, B. S., Rowe, C. A., Taylor, S. F., Wyatt, L. S., Moss, B., Small, P. A., Jr., “Oral immunization with a replication-deficient recombinant vaccinia virus protects mice against influenza,” J. Virol. 70:6418-24, 1996.

Brandt, S. J., Bodine, D. M., Dunbar, C. E., Nienhuis, A. W., “Dysregulated interleukin 6 expression produces a syndrome resembling Castleman's disease in mice,” J. Clin. Invest. 86:592-9, 1990.

Brown, G. B. and J. K. McMillen, “MaxiVac™-Flu: evaluation of the safety and efficacy of a swine influenza vaccine,” Am. J. Trop. Med. Hyg. 50:3-9, 1994.

Campitelli, L., I. Donatelli, E. Foni, M. R. Castrucci, C. Fabiani, Y. Kawaoka, S. Krauss, and R. G. Webster, “Continued evolution of H1N1 and H3N2 influenza viruses in pigs in Italy,” Virology 232:310-318, 1997.

Castrucci, M. R., I. Donatelli, L. Sidoli, G. Barigazzi, Y. Kawaoka, and R. G. Webster, “Genetic reassortment between avian and human influenza viruses in Italian pigs,” Virology 193:503-506, 1993.

Chow, Y. H., Huang, W. L., Chi, W. K., Chu, Y. D., Tao, M. H., “Improvement of hepatitis B virus DNA vaccines by plasmids coexpressing hepatitis B surface antigen and interleukin-2,” J. Virol. 71:169-78, 1997.

Claas, E. C. J., Y. Kawaoka, J. C. de Jong, N. Masurel, R. G. Webster, “Infection of children with avian-human reassortant influenza virus from pigs in Europe,” Virology 204:453-457, 1994.

Clements, M. L., O'Donnell, S., Levine, M. M., Chanock, R. M., Murphy, B. R., “Dose response of A/Alaska/6/77 (H3N2) cold-adapted reasortant vaccine virus in adult volunteers: role of local antibody in resistance to infection with vaccine virus,” Infect. Immun. 40:1044-51, 1983.

Dasco, C. C., R. B. Couch, H. R. Six, J. F. Young, J. M. Quarles, J. A. Kasel, “Sporadic occurrence of zoonotic swine influenza virus infections,” J. Clin. Microbiol. 20:833-835, 1984.

Deck, R. R., DeWitt, C. M., Donnelly, J. J., Liu, M. A., Ulmer, J. B., “Characterization of humoral immune responses induced by an influenza hemagglutinin DNA vaccine,” Vaccine 15:71-8, 1997.

de Jong, J. C., Paccaud, M. F., de Ronde-Verloop, F. M., Huffels, N. H., Verwei, C., Weijers, T. F., Bangma, P. J., van Kregten, E., Kerckhaert, J. A. M., Wicki, F., Wunderli, W., “Isolation of swine-like influenza A (H1N1) viruses from men in Switzerland and the Netherlands,” Annu. Inst. Pasteur/Virol. 139:429-437, 1988.

Donnelly, J. J., Ulmer, J. B., Liu, M. A., “Immunization with DNA,” J. Immunol. Methods 176:145-52, 1994.

Donnelly, J. J., et al., Life Sci. 60:163-72, 1996.

Eason, R. J. and M. D. Sage, “Deaths from influenza A, subtype H1N1, during the 1979 Auckland epidemic,” New Zealand Medical Journal 91:129-131, 1980.

Easterday, B. C. and V. S. Hinshaw, “Swine influenza,” Diseases of Swine, In A. D. Leman, B. E. Straw, W. L. Mengeling, S. D. D'Allaire, D. J. Taylor (eds.), Iowa State Press, Ames, p. 349-357, 1992.

Finkelman, F. D., Holmes, J., Katona, I. M., et al., “Lymphokine control of in vivo immunoglobulin isotype selection,” Annu. Rev. Immunol. 8:303-33, 1990.

Fynan, E. F., Webster, R. G., Fuller, D. H., Haynes, J. R., Santoro, J. C., Robinson, H. L., “DNA vaccines: Protective immunizations by parenteral, mucosal, and gene-gun inoculations,” Proc. Natl. Acad. Sci. USA 90:11478-82, 1993.

Fynan, E. F., Webster, R. G., Fuller, D. H., Haynes, J. R., Santoro, J. C., Robinson, H. L., “DNA vaccines: A novel approach to immunization,” Int. J. Immunopharmacol. 17:79-83, 1995.

Geissler, M., Gesien, A., Tokushige, K., Wands, J. R., “Enhancement of cellular and humoral immune responses to hepatitis C virus core protein using DNA-based vaccines augmented with cytokine-expressing plasmids,” J. Immunol. 158:1231-7, 1997.

Guan, Y., K. F. Shortridge, S. Krauss, P. H. Li, Y. Kawaoka, R. G. Webster, “Emergence of avian H1N1 influenza viruses in pigs in China,” J. Virol. 70:8041-8046, 1996.

Hannant, D., Mumford, J. A. and Jesset, D. M., “Duration of circulating antibody and immunity following infection with equine influenza virus,” Vet. Rec. 122:125-8, 1988.

Haynes, J. R., McCabe, D. E., Swain, W. F., Widera, G., Fuller, J. T., “Particle-mediated nucleic acid immunization,” J. Biotechnol. 44:37-42, 1996.

Hengge, U. R., Chan, E. F., Foster, R. A., Walker, P. S., Vogel, J.C., “Cytokine gene expression in epidermis with biological effects following injection of naked DNA,” Nature Genetics 10:161-6, 1996.

Hinshaw, V. S., Bean, Jr., W. J., Webster, R. G., Easterday, B. C., “The prevalence of influenza viruses in swine and the antigenic and genetic relatedness of influenza viruses from man and swine,” Virology 84:51-62, 1978.

Holmgren, J., Czerkinsky, C., Lycke, N., Svennerholm, A.-M., “Mucosal immunity: implications for vaccine development,” Immunobiol. 184:157-79, 1992.

Ito, T. and Y. Kawaoka. 1998. Unpublished results.

Justewicz, D. M., Doherty, P. C., Webster, R. G., “The B-cell response in lymphoid tissue of mice immunized with various antigenic forms of the influenza virus hemagglutinin,” J. Virol. 69:5414-5421, 1995.

Kida, H., Ito, T., Yasuda, J., Shimizu, Y., Itakura, C., Shortridge, K. F., Kawaoka, Y., Webster, R. G., “Potential for transmission of avian influenza viruses to pigs,” J. Gen. Virol. 75:2183-2188, 1994.

Kimura, K., Adlakha, A., Simon, P. M., “Fatal case of swine influenza virus in an immunocompetent host,” Mayo Clin. Proc. 73:243-245, 1998.

Larsen, D. L., Dybdahl-Sissoko, N. R., McGregor, M. W., Drap, K., Neumann, V., Swain, W. F., Lunn, D. P., Olsen, C. W., “Coadministration of DNA encoding interleukin-6 and hemagglutinin confers protection from influenza virus challenge in mice,” J. Virol. 72:1704-1708, 1998.

Larsen, D. L., Dybdahl-Sissoko, N. R., Olsen, C. W., “Comparison of the infectivity of A/Equine/Kentucky/1/81 and A/Swine/Indiana/1726/88 for mice,” Unpublished results, 1998.

Larsen, D. L. and C. W. Olsen, “The mediastinal lymph node is a prominent site of virus-specific primary and memory B cell responses following influenza virus infection in mice and pigs,” Unpublished results, 1998.

Liew, F. Y., Russell, S. M., Appleyard, G., Brand, C. M., Beale, J., “Cross-protection in mice infected with influenza A virus bt the respiratory route is correlated with local IgA antibody rather than serum antibody or cytotoxic T cell reactivity,” Eur. J. Immunol. 14:350-6, 1984.

Lin, R., Tarr, P. E., Jones, T. C., “Present status of the use of cytokines as adjuvants with vaccines to protect against infectious diseases,” Clin. Infect. Dis. 21:1439-49, 1995.

Liu, M. A., Ann. NY Acad. Sci. 772:15-20, 1995 (entire volume was devoted to DNA vaccination).

Lofthouse, S. A., Andrews, A. E., Nash, A. D., Bowles, V. M., “Humoral and cellular responses induced by intradermally administered cytokine and conventional adjuvants,” Vaccine 13:1131-7, 1995.

Macklin, M. D., McCabe, D., McGregor, M. W., Neumann, V., Meyer, T., Callan, R., Hinshaw, V. S., Swain, W. F., “Immunization of pigs with a particle-mediated DNA vaccine to influenza A virus protects against challenge with homologous virus,” J. Virol. 72:1491-1496, 1998.

Madic, J., Martinovic, S., Naglic, T., Hajsig, D., Cvetnic, S., “Serological evidence for the presence of A/equine-1 influenza virus in unvaccinated horses in Croatia,” Vet. Rec. 138:68, 1996.

McGhee, J. R., Kiyono, H., “Mucosal immunity to vaccines: current concepts for vaccine development and immune response analysis. In: Ciardi J. E., eds. Genetically engineered vaccines. New York: Plenum Press, pp. 3-12, 1992.

Mosmann, T. R. and S. Sad, “The expanding universe of T-cell subsets: Th1, Th2 and more,” Immunol. Today 17:138-46, 1996.

Mumford, J., “Progress in the control of equine influenza.” In: Plowright W, Rossdale P D, Wade J F, eds. Equine Infectious Diseases VI. Proceedings of the Sixth International Conference. Newmarket: R and W Publications, 207-17, 1992.

Mumford, J. A., Jessett, D., Dunleavy, U., Wood, J., Hannant, D., Sundquist, B., Cook, R. F., “Antigenicity and immunogenicity of experimental equine influenza ISCOM vaccines,” Vaccine 12:857-63, 1994.

Murphy, B. R. and R. G. Webster, “Orthomyxoviruses,” In: Fields B N, Knipe D M, Howley P M, et al., editors. Field's Virology. 3rd ed. Philadelphia: Lippincott-Raven Publishers, pp. 1397-445, 1996.

Nakajima, K., Nakajima, S., Shortridge, K. F., Kendal, A. P., “Further genetic evidence for maintenance of early Hong Kong-like influenza A (H3N2) strains in swine until 1976,” Virology 116:562-572, 1982.

Noble, S., McGregor, M. S., Hinshaw, V. S., “Cloning of the full-length hemagglutinin gene of A/Swine/Indiana/1726/88 influenza virus,” Unpublished results, 1993.

Noll, A. and I. B. Autenrieth, “Immunity against Yersinia enterocolitica by vaccination with Yersinia HSP60 immunostimulating complexes or Yersinia HSP60 plus interleukin-12,” Infect. Immun. 64:2955-61, 1996.

Olsen, C. W., McGregor, M. W., Cooley, A. J., Schantz, B., Hotze, B., Hinshaw, V. S., “Antigenic and genetic analysis of a recently isolated H1N1 swine influenza virus,” Am. J. Vet. Res. 54:1630-6, 1993.

Olsen, C. W., McGregor, M. W., Dybdahl-Sissoko, N., et al., “Immunogenicity and efficacy of baculovirus-expressed and DNA-based equine influenza virus hemagglutinin vaccines in mice,” Vaccine 15:1149-1156, 1997.

Ottis, K., Sidoli, L., Bachman, P. A., Webster, R. G., Kaplan, M. M., “Human influenza A viruses in pigs: isolation of a H3N2 strain antigenically related to A/England/42/72 and evidence for continuous circulation of human viruses in the pig population,” Arch. Virol. 73:103-108, 1982.

Palmer, D. F., Dowdle, W. R., Coleman, M. T., Schild, G. C., “Advanced laboratory techniques for influenza diagnosis,” 6th ed. United States Department of Health, Education and Welfare Immunology Series, 1975.

Patriarca, P. A., Kendal, A. P., Zakowski, P. C., Cox, N. J., Trautman, M. S., Cherry, J. D., Auervach, D. M., McCusker, J., Belliveau, R. R., Kappus, K. D., “Lack of significant person-to-person spread of swine influenza-like virus following fatal infection of an immunocompromised child,” Am. J. Epidemiol. 119:152-158, 1984.

Pensaert, M., Ottis, K., Vandeputte, J., Kaplan, M. M., Bachmann, P. A., “Evidence for the natural transmission of influenza A virus from wild ducks to swine and its potential importance for man,” Bull. World Health Org. 59:75-78, 1981.

Pertmer, T. M., Eisenbraun, M. D., McCabe, D., Prayaga, S. K., Fuller, D. F., Haynes, J. R., “Gene gun-based nucleic acid immunization: Elicitation of humoral and cytotoxic T. lymphocyte responses following epidermal delivery of nanogram quantities of DNA,” Vaccine 13:1427-30, 1995.

Pockley, A. G. and P. C. Montgomery, “In vivo adjuvant effect of interleukins 5 and 6 on rat tear IgA antibody responses,” Immunology 73:19-23, 1991.

Ramsay, A. J. and M. Kohonsen-Corish, “Interleukin-5 expressed by a recombinant virus vector enhances specific mucosal IgA responses in vivo,” Eur. J. Immunol. 23:3141-5, 1993.

Ramsay, A. J., Husband, A. J., Ramshaw, I. A., Bao, S., Matthaei, K. I., Koehler, G., Kopf, M., “The role of interleukin-6 in mucosal IgA antibody responses in vivo,” Science 264:561-3, 1994.

Reed, L. J. and H. Muench, “Simple method of estimating 50 percent endpoints,” Am. J. Hyg. 27:493-7, 1938.

Renegar, K. B. and P. A. Small, Jr., “Passive transfer of local immunity to influenza virus infection by IgA antibody,” J. Immunol. 146:1972-8, 1991.

Renegar, K. B. and P. A. Small, Jr., “Immunoglobulin A mediation of murine nasal anti-influenza virus immunity,” J. Virol. 65:2146-8, 1991.

Robinson, H. L., Lu, S., Feltquate, D. M., et al., “DNA vaccines,” AIDS Res. Hum. Retroviruses 12:455-7, 1996.

Rota, P. A., Rocha, E. P., Harmon, M. W., Hinshaw, V. S., Sheerar, M. G., Kawaoka, Y., Smith, T. L., “Laboratory characterization of a swine influenza virus isolated from a fatal case of human influenza,” J. Clin. Microbiol. 27:1413-1416, 1989.

Sakaguchi, M., Nakamura, H., Sonoda, K., Hamada, F., Hirai, K., “Protection of chickens from Newcastle disease by vaccination with a linear plasmid DNA expressing the F protein of Newcastle disease virus,” Vaccine 14:747-52, 1996.

Scholtissek, C., Burger, H., Bachmann, P. A., Hannoun, C., “Genetic relatedness of hemagglutinins of the H1 subtype of influenza A viruses isolated from swine and birds,” Virology 129:521-523, 1983.

Scholtissek, C., Burger, H., Kistner, O., Shortridge, K., “The nucleoprotein as a possible major factor in determining host specificity of influenza H3N2 viruses,” Virology 147:287-294, 1985.

Scholtissek, C. and E. Naylor, “Fish farming and influenza pandemics,” Nature 331:215, 1988.

Shu, L. L., Lin, Y. P., Wright, S. M., Shortridge, K. F., Webster, R. G., “Evidence for interspecies transmission and reassortment of influenza A viruses in pigs in Southern China,” Virology 202:825-833, 1994.

Singh, G., “Equine influenza 1987, post-epidemic serological study in North India,” J. Eq. Vet. Sci. 12:342-4, 1996.

Sun, W. H., Burkholder, J. K., Sun, J., et al., “In vivo cytokine gene transfer by gene gun reduces tumor growth in mice,” Proc. Natl. Acad. Sci. USA 92:2889-93, 1995.

Takase, H., Murakami, Y., Endo, A., Ikeuchi, T., “Antibody responses and protection in mice immunized orally against influenza virus,” Vaccine 14:1651-6, 1996.

Tamura, S., Funato, H., Hirabayashi, Y., Suzuki, Y., Nagamine, T., Aizawa, C., Kurata, T., “Cross-protection against influenza A virus infection by passively transferred respiratory tract IgA antibodies to different hemagglutinin molecules,” Eur. J. Immunol. 21:1337-44, 1991.

Top, F. H. and P. K. Russell, “Swine influenza at Fort Dix, N.J. IV. Summary and speculation,” J. Infect. Dis. 136:S376-S380, 1977.

Traub-Dargatz, J. L., Salman, M. D., Voss, J. L., “Medical problems of adult horses, as ranked by equine practitioners,” J. Am. Vet. Med. Assoc. 198:1745-7, 1991.

Ulmer, J. B., Donnelly, J. J., Parker, S. E., et al., “Heterologous protection against influenza by injection of DNA encoding a viral protein,” Science 259:1745-9, 1993.

Ulmer, J. B., Deck, R. R., DeWitt, C. M., Friedman, A., Donnelly, J. J., Liu, M. A., “Protective immunity by intramuscular injection of low doses of influenza virus DNA vaccines,” Vaccine 12:1541-4, 1994.

Ulmer, J. B., et al., Curr. Opin. Immunol. 8:531-6, 1996.

Van Snick, J., “Interleukin-6: an overview,” Annu. Rev. Immunol. 8:253-78, 1990.

Webster, R. G., Bean, W. J., Gorman, O. T., Chambers, T. M., Kawaoka, Y., “Evolution and ecology of influenza A viruses,” Microbiol. Rev. 56:152-179, 1992.

Webster, R. G., Fynan, E. F., Santoro, J. C., Robinson, H., “Protection of ferrets against influenza challenge with a DNA vaccine to the haemagglutinin,” Vaccine 12:1495-8, 1994.

Wentworth, D., Xian, X., Cooley, A. J., McGregor, M. W., Hinshaw, V. S., Cox, N., “An influenza A (H1N1) virus closely related to swine influenza responsible for a fatal case of human influenza,” J. Virol. 68(4):2051-2058, 1994.

Wentworth, D. E., McGregor, M. W., Macklin, M. D., Neumann, V., Hinshaw, V. S., “Transmission of swine influenza virus to humans after exposure to experimentally infected pigs,” J. Infect. Dis. 175:7-15, 1997.

Whalen, R. G., “DNA vaccines for emerging infectious diseases: What if”? Emerg. Infect. Dis. 2:168-75, 1996.

Xiang, Z. and H. C. Ertl, “Manipulation of the immune response to a plasmid-encoded viral antigen by coinoculation with plasmids expressing cytokines,” Immunity 2:129-35, 1995.

Zhou, N., He, S., Zhang, T., Zou, W., Shu, L., Sharp, G. B., Webster, R. G., “Influenza infection in humans and pigs in southeastern China,” Arch. Virol. 141:649-661, 1996.

We claim:

1. A method for eliciting an enhanced immune response against a selected antigen in a mammalian subject, said method comprising administering to said subject a composition comprising a first nucleic acid sequence encoding interleukin-6 and a second nucleic acid sequence encoding the selected antigen, wherein said first and second nucleic acid sequences are operably linked to control sequences which direct the expression thereof in said subject.

2. The method of claim 1, wherein the first and second sequences are present in a single nucleic acid construct.

3. The method of claim 1, wherein the enhanced immune response is characterized by the generation of an enhanced antibody response against said antigen.

4. The method of claim 1, wherein the enhanced immune response is characterized by the generation of an enhanced T-cell response against said antigen.

5. The method of claim 1, wherein the composition is a therapeutic vaccine composition.

6. The method of claim 1, wherein the composition is a prophylatic vaccine composition.

7. The method of claim 3, wherein the antibody response is protective.

8. The method of claim 1, wherein the selected antigen is a viral antigen.

9. The method of claim 1, wherein the selected antigen is a bacterial antigen.

10. The method of claim 1, wherein the selected antigen is a tumor antigen.

11. The method of claim 1, wherein the composition is administered using particle-mediated delivery techniques.

12. The method of claim 1, wherein the composition is administered to a target skin site in said subject.

13. The method of claim 1, wherein the composition is administered to a target mucosal surface in said subject.

14. The method of claim 13, wherein the composition is administered to the tongue.

15. The method of claim 1, wherein the viral antigen is derived from a virus selected from the group consisting of influenza viruses, rotaviruses, herepes viruses, and hepatitis viruses.

16. The method of claim 15, wherein the virus is an influenza virus.

17. The method of claim 15, wherein the virus is a hepatitis virus.
